(19) United States
(12) Patent Application Publication
(10) Pub. No.: US 2022/0192850 A1
(43) Pub. Date: Jun. 23, 2022

(54) STENT AND AFFIXING METHOD FOR ELEMENT WIRES IN STENT

(71) Applicant: PENTAS Inc., Tokyo (JP)

(72) Inventor: Makoto NISHIGISHI, Tokyo (JP)

(21) Appl. No.: 17/606,341

(22) PCT Filed: Jun. 5, 2020
(86) PCT No.: PCT/JP2020/022298

§ 371 Date: Oct. 25, 2021

(30) Foreign Application Priority Data
Jul. 9, 2019 (JP) 2019-127645

(51) Int. Cl. A61F 2/89; A61F 2/88

(52) U.S. Cl. CPC A61F 2/89; A61F 2210/0014; A61F 2210/0004; A61F 2/88

(57) ABSTRACT

To fix an intersection at a stent, a stent is configured such that multiple strands are woven in a spiral shape, two crossing strands are fixed by a fixing material having rubber elasticity at an intersection between the strands.

### TECHNICAL FIELD

The present invention relates to a stent and a stent strand fixing method.

### BACKGROUND ART

The following medical stent has been known. This stent is formed in such a manner that multiple strands are woven in a spiral shape (see Patent Literature 1).

### CITATION LIST

### Patent Literature

PATENT LITERATURE 1: JP-A-2012-223209

### SUMMARY OF INVENTION

### Problems to be Solved by Invention

Generally, strands are not fixed, but are independent of each other at a stent formed by weaving of the strands. Thus, there is a probability that entanglement of both ends of the stent occurs upon production of the stent and the shape of the stent is deformed accordingly. Moreover, in some cases, displacement of the strands occurs in a catheter upon delivery of the stent. Further, in some cases, expansive force is insufficient only by expansion utilizing the elasticity of the strands upon expansion of the stent. However, no study has been typically conducted on the technique of solving these problems.

### Solutions to Problems

According to a first aspect of the present invention, a stent includes: multiple strands woven in a spiral shape, in which at an intersection between crossing two of the strands, the crossing two of the strands are fixed by a fixing material having rubber elasticity.

According to a second aspect of the present invention, in the stent according to the first aspect, a strand intersection positioned in a vicinity of each end of the stent is fixed by the fixing material.

According to a third aspect of the present invention, in the stent according to the first aspect, a strand intersection positioned in a vicinity of each end of the stent and a strand intersection positioned at a center portion of the stent are fixed by the fixing material.

According to a fourth aspect of the present invention, in the stent according to the first aspect, all strand intersections are fixed by the fixing material.

According to a fifth aspect of the present invention, in the stent according to any one of the first to fourth aspects, the fixing material is a material having the rubber elasticity, such as resin having a shape-memory property or bioabsorbable resin.

According to a sixth aspect of the present invention, a method for fixing ones of multiple strands of a stent is configured such that the multiple strands are woven in a spiral shape, the method including: fixing, by a fixing material having rubber elasticity, crossing two of the strands at an intersection between the crossing two of the strands.

According to a seventh aspect of the present invention, in the stent strand fixing method according to the sixth aspect, a strand intersection positioned in a vicinity of each end of the stent is fixed by the fixing material.

According to an eighth aspect of the present invention, in the stent strand fixing method according to the sixth aspect, a strand intersection positioned in a vicinity of each end of the stent and a strand intersection positioned at a center portion of the stent are fixed by the fixing material.

According to a ninth aspect of the present invention, in the stent strand fixing method according to the sixth aspect, all strand intersections are fixed by the fixing material.

According to a tenth aspect of the present invention, in the stent strand fixing method according to any one of the sixth to ninth aspects, the fixing material is a material having the rubber elasticity, such as resin having a shape-memory property or bioabsorbable resin.

### Effects of Invention

According to the present invention, the two crossing strands are, at the strand intersection, fixed by the fixing material having the rubber elasticity, and therefore, occurrence of entanglement of both ends of the stent upon production thereof can be prevented. Moreover, occurrence of displacement of the strands in a catheter upon delivery of the stent can be also prevented. Further, even in a case where expansive force is insufficient only by expansion utilizing the elasticity of the strands upon expansion of the stent, expansion of the stent is assisted by the fixing material having the rubber elasticity so that the stent can be uniformly expanded.

### BRIEF DESCRIPTION OF DRAWINGS

FIG. 1 is a view schematically showing the shape of a stent.

FIG. 2 is a view schematically showing a state in which entanglement has occurred at both ends of the stent.

FIG. 3 is a view schematically showing a state in which markers are displaced.

FIG. 4 is a view schematically showing a state in which two crossing strands are fixed at a strand intersection.

FIG. 5 is a view schematically showing a fixed portion using a fixing material.

FIG. 6 is a view showing a variation of the strand fixing portions.

FIG. 7 is a first view showing a variation of a strand fixing method.

FIG. 8 is a second view showing a variation of the strand fixing method.

FIG. 9 is a third view showing a variation of the strand fixing method.

### DESCRIPTION OF EMBODIMENTS

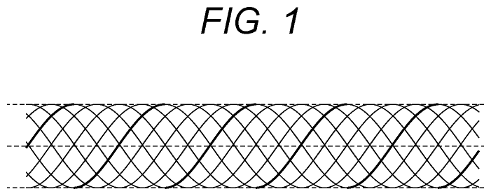

FIG. 1 is a view schematically showing the shape of a stent. In the present embodiment, a stent configured such that multiple strands are woven in a spiral shape as shown in FIG. 1. As the strand, a strand made of metal such as stainless steel, cobalt-chromium alloy (Co—Cr alloy), or nickel-titanium alloy (Ni—Ti alloy) is used, for example.

The stent is formed in such a manner that the multiple metal strands are woven in the spiral shape as described above, and there are multiple types of the number of strands forming the stent. Examples include a stent having a 16-strand structure in which 16 strands are woven, a stent having a 24-strand structure in which 24 strands are woven, and a stent having a 32-strand structure in which 32 strands are woven. Note that FIG. 1 shows the stent having the 16-strand structure.

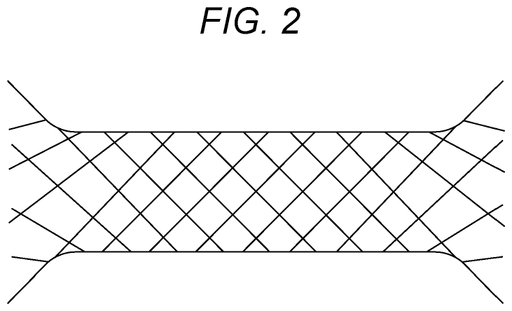

Generally, in the stent formed in such a manner that the multiple metal strands are woven in the spiral shape as described above, both end sides of the strands are not fixed and are independent of each other. Thus, as shown in FIG. 2, there is a probability that entanglement is caused on these both end sides upon production of the stent. When the stent in which entanglement is caused on both end sides as described above, the shape of the stent upon expansion thereof is deformed.

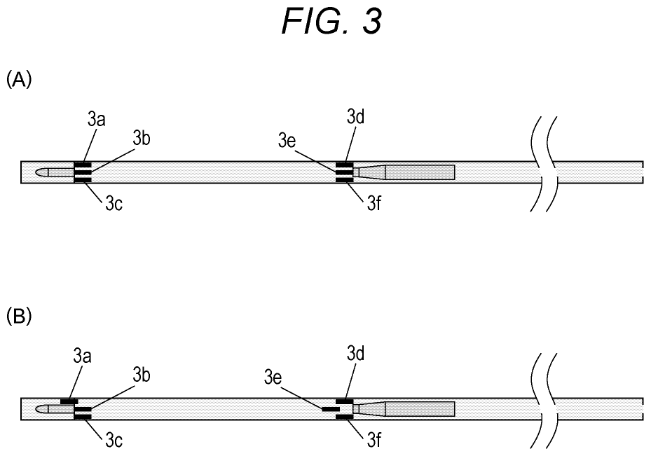

Moreover, displacement of the strands is caused in a catheter upon delivery of the stent in some cases, and in this case, there is a probability that markers fixed to both end sides of the strands are displaced. For example, markers 3a to 3f are not displaced as shown in FIG. 3(A) in a normal state, and on the other hand, the marker 3a and the marker 3e are displaced due to displacement of the strands in an example shown in FIG. 3(B).

Further, in some cases, expansive force is insufficient only by expansion utilizing the elasticity of the strands upon expansion of the stent. That is, at a center portion (an intermediate portion) of the stent, the expansive force is also applied from both sides in addition to the expansive force of the strands at such a center portion, and therefore, sufficient expansive force can be obtained. On the other hand, at both end portions of the stent, the expansive force is applied from a center side, but no expansive force is applied from end sides. Thus, sufficient expansive force is not obtained, and therefore, there is a probability that a problem such as insufficient expansion or non-uniform expansion of the end portions of the stent is caused.

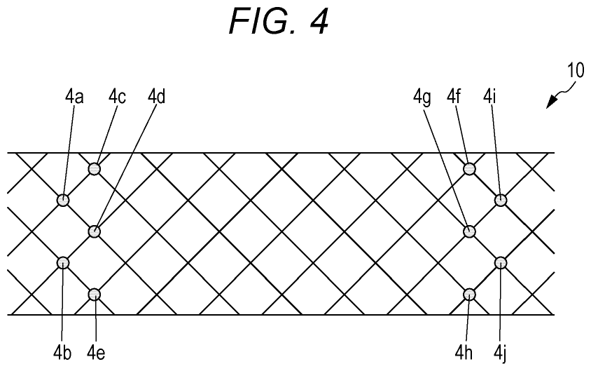

For solving these problems, a stent 10 is, in the present embodiment, configured such that two crossing strands are fixed at an intersection between the strands as shown in FIG. 4. Note that FIG. 4 is a view schematically showing a state in which the strand intersections are fixed using fixing materials 4a to 4j at the expanded stent and shows a state in which the vicinity of both ends of the stent 10 is targeted for fixing of the intersections. Note that in the case of fixing the intersections positioned in the vicinity of both ends of the stent 10, as shown in FIG. 4, intersections positioned at second and third columns from the end of the stent 10 may be fixed.

As described above, the intersections in the vicinity of both ends of the stent 10 are fixed, and therefore, entanglement of both ends of the stent as shown in FIG. 2 can be prevented. Moreover, occurrence of displacement of the strands in the catheter upon stent delivery can be also prevented, and therefore, displacement of the markers as shown in FIG. 3 can be also prevented.

Further, in the stent 10 of the present embodiment, the fixing materials 4a to 4j made of a material having rubber elasticity are used as fixing members for fixing the strand intersections. As the fixing material having the rubber elasticity, resin having shape-memory properties or bioabsorbable resin, such as silicon, may be used, for example. Thus, the fixing materials can contract the stent and deform the material, and can improve the expandability of the stent by means of restoring force provided by the rubber elasticity upon stent expansion.

Specifically, as shown in FIG. 4, the intersections in the vicinity of both ends of the stent 10 are fixed by the fixing materials 4a to 4j having the rubber elasticity, and therefore, the expandability of both stent end portions at which sufficient expansive force might not be able to be obtained only by the expansive force of the strands can be improved.

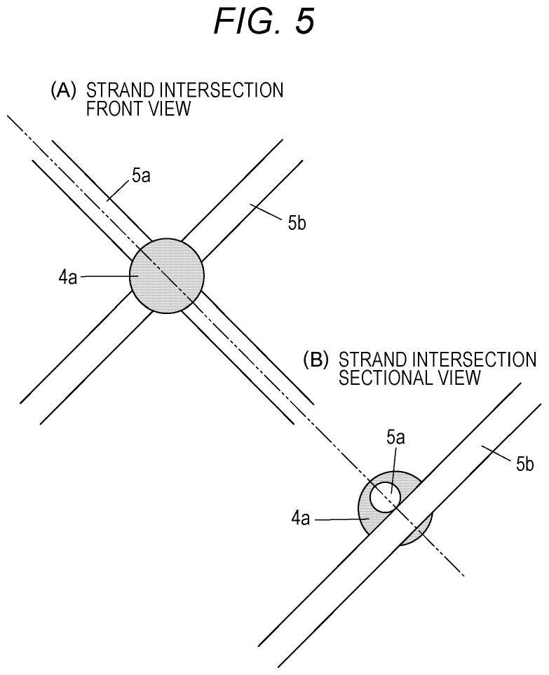

FIG. 5 shows schematic views of a state in which an intersection between two strands is fixed using the fixing material 4a. As shown in FIG. 5, at an intersection between a strand 5a and a strand 5b, these two strands are covered with the fixing material 4a in a state in which the strand 5a and the strand 5b overlap with each other, and in this manner, these two strands are fixed. In FIG. 5, FIG. 5(A) is a front view of the strand intersection, and this figure shows that the two strands are fixed by the fixing material 4a at the intersection between the strand 5a and the strand 5b. Moreover, FIG. 5(B) is a sectional view of the strand intersection, and this figure shows that the two strands are covered with the fixing material 4a in a state in which the strand 5a and the strand 5b overlap with each other.

According to the present embodiment described above, the following features and advantageous effects can be obtained.

(1) In the stent configured such that the multiple strands are woven in the spiral shape, the two crossing strands are, at the intersection between the strands, fixed by the fixing material having the rubber elasticity. With this configuration, entanglement of both stent ends can be prevented. Moreover, occurrence of displacement of the strands in the catheter upon stent delivery can be also prevented. Further, the strand intersection is fixed by the fixing material having the rubber elasticity. Thus, the fixing material can contract the stent and deform the material, and can improve the expandability of the stent by means of the restoring force provided by the rubber elasticity upon stent expansion.

(2) The strand intersections positioned in the vicinity of both ends of the stent are fixed with the fixing materials. With this configuration, the expandability of both stent end portions at which sufficient expansive force might not be able to be obtained only by the expansive force of the strands can be improved.

Variations

Note that the stent of the above-described embodiment can be modified as follows.

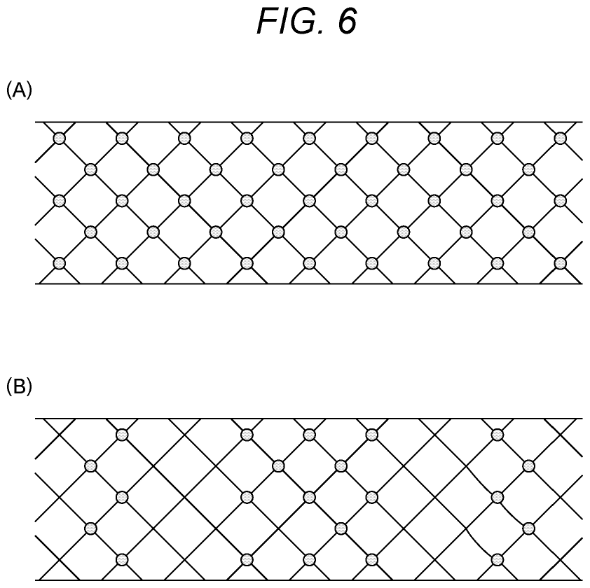

(1) In the above-described embodiment, the example where the intersections positioned at the second and third columns from the end of the stent 10 are fixed as shown in FIG. 4, i.e., the intersections in the vicinity of both ends of the stent 10 are fixed, has been described. However, as long as the object of the present invention can be achieved, the position of the intersection at which the strands are fixed is not limited to the vicinity of both ends. For example, as shown in FIG. 6(A), all strand intersections may be fixed by fixing materials. Alternatively, as shown in FIG. 6(B), strand intersections positioned in the vicinity of both ends of the stent and strand intersections positioned at the center portion of the stent may be fixed by fixing materials. Since the strand intersections are fixed in any of the examples shown in FIGS. 6(A) and 6(B), entanglement of both stent ends and displacement of the strands in the catheter upon stent delivery can be prevented as in the above-described embodiment. Moreover, since the strands are fixed at the intersections close to both ends of the stent in any of these examples, the expandability of both stent ends at which the expansive force tends to be insufficient can be improved.

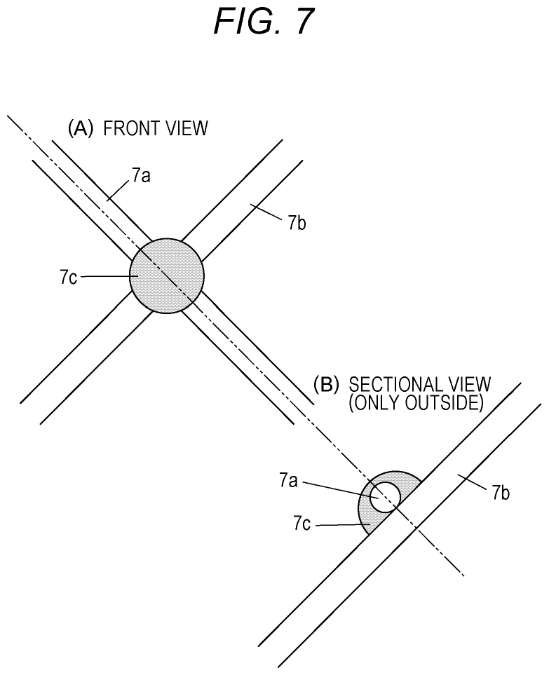

(2) In the above-described embodiment, the example where the two strands are covered and fixed with the fixing material in a state in which the two strands overlap with each other as shown in FIG. 5 has been described. However, the method for fixing the strands by the fixing material is not limited to such a method. For example, as shown in FIG. 7, at an intersection between a strand 7a and a strand 7b, only a front side (a stent outer side) of these two strands may be fixed by a hemispherical fixing material 7c in a state in which the strand 7a and the strand 7b overlap with each other. In FIG. 7, FIG. 7(A) is a front view of the strand intersection, and this figure shows that the two strands are fixed by the fixing material 7c at the intersection between the strand 7a and the strand 7b. Moreover, FIG. 7(B) is a sectional view of the strand intersection, and this figure shows that the outside, i.e., the front side, of the two strands is fixed by the hemispherical fixing material 7c in a state in which the strand 7a and the strand 7b overlap with each other.

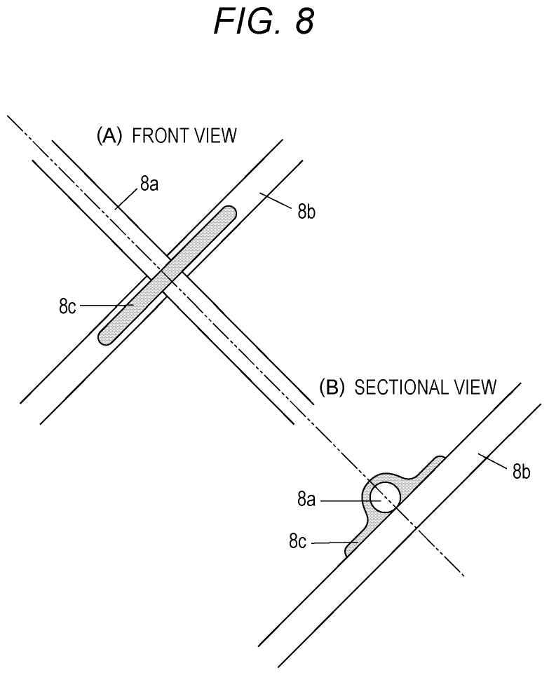

Alternatively, as shown in FIG. 8, at an intersection between a strand 8a and a strand 8b, these two strands may be fixed by an elongated fixing material 8c along the strand 8b positioned on the lower side in a state in which the strand 8a and the strand 8b overlap with each other. In FIG. 8, FIG. 8(A) is a front view of the strand intersection, and this figure shows that the two strands are fixed by the elongated fixing material 8c along the strand 8b at the intersection between the strand 8a and the strand 8b. Moreover, FIG. 8(B) is a sectional view of the strand intersection, and this figure shows that the outside of the two strands is fixed by the fixing material 8c in a state in which the strand 8a and the strand 8b overlap with each other.

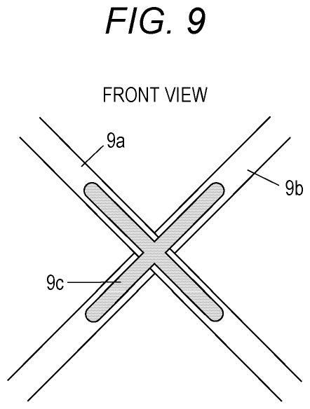

Alternatively, as shown in FIG. 9, at an intersection between a strand 9a and a strand 9b, these two strands may be fixed by a cross-shaped fixing material 9c along the strand 9a and the strand 9b in a state in which the strand 9a and the strand 9b overlap with each other. In any method, the above-described object of the present invention can be achieved using the material having the rubber elasticity as the fixing material.

Note that the present invention is not limited to the configuration in the above-described embodiment as long as the characteristic functions of the present invention are not degraded. Moreover, the above-described embodiment and multiple variations may be combined with each other.

The disclosure of the following basic application claiming the priority is incorporated herein by reference:

Japanese Patent Application No. 2019-127645 (filed on Jul. 9, 2019)

### LIST OF REFERENCE SIGNS

10 Stent
3a to 3f Marker
4a to 4j, 7c, 8c, 9c Fixing Material
5a, 5b, 7a, 7b, 8a, 8b, 9a, 9b Strand

1. A stent comprising:
multiple strands woven in a spiral shape,
wherein at an intersection between crossing two of the strands, the crossing two of the strands are fixed by a fixing material having rubber elasticity.

2. The stent according to claim 1, wherein
a strand intersection positioned in a vicinity of each end of the stent is fixed by the fixing material.

3. The stent according to claim 1, wherein
a strand intersection positioned in a vicinity of each end of the stent and a strand intersection positioned at a center portion of the stent are fixed by the fixing material.

4. The stent according to claim 1, wherein
all strand intersections are fixed by the fixing material.

5. The stent according to claim 1, wherein
the fixing material is a material having the rubber elasticity, such as resin having a shape-memory property or bioabsorbable resin.

6. A method for fixing ones of multiple strands of a stent configured such that the multiple strands are woven in a spiral shape, comprising:
fixing, by a fixing material having rubber elasticity, crossing two of the strands at an intersection between the crossing two of the strands.

7. The stent strand fixing method according to claim 6, wherein
a strand intersection positioned in a vicinity of each end of the stent is fixed by the fixing material.

8. The stent strand fixing method according to claim 6, wherein
a strand intersection positioned in a vicinity of each end of the stent and a strand intersection positioned at a center portion of the stent are fixed by the fixing material.

9. The stent strand fixing method according to claim 6, wherein
all strand intersections are fixed by the fixing material.

10. The stent strand fixing method according to claim 6, wherein
the fixing material is a material having the rubber elasticity, such as resin having a shape-memory property or bioabsorbable resin.
